(19) United States
(12) Patent Application Publication
(10) Pub. No.: US 2004/0162337 A1
(43) Pub. Date: Aug. 19, 2004

(54) Chemical compounds having therapeutic activities in treating cancer

(76) Inventors: Wang Fun Fong, Hong Kong (CN); Yiu Cheong Jimmy Wu, Hong Kong (CN)

Correspondence Address: INTELLECTUAL PROPERTY GROUP, FREDRIKSON & BYRON, P.A., 4000 PILLSBURY CENTER, 200 SOUTH SIXTH STREET, MINNEAPOLIS, MN, 55402, US

(21) Appl. No.: 10/354,443

(22) Filed: Jan. 30, 2003

(51) Int. Cl. A61K 31/366; C07D 493/02

(52) U.S. Cl. 514/455; 549/282

(57) ABSTRACT

Chemical compounds of the general formula

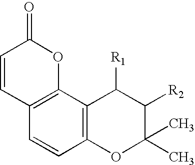

in which R1 and R2 which may be the same or different, each represents a saturated or unsaturated acid. The present invention is related to the previously mentioned compounds, the compounds therapeutic activity in treating cancer, and a method of isolating such chemical compounds from natural and synthetic sources.

### FIELD OF INVENTION

The present invention is primarily concerned with a set of related chemical compounds, each of which having therapeutic activity in treating cancer, and a method of isolating such chemical compounds from natural and synthetic sources.

### BACKGROUND OF THE INVENTION

A variety of chemical compounds or combination thereof have been proposed to possess activity in suppressing and/or causing apoptosis in cancer cells. While some of these chemical compounds do to an extent possess certain types of the anti-cancer activity, it has often been found that when one or more conventional cancer therapeutic drugs having these chemical compounds have been used for an extended period of time for treating cancer, some cancer cells will often become multi-drug resistant (MDR). This is of course undesirable as relapses would occur, and the treatment of relapses will become more difficult. One of the traditional approaches to tackle this problem is to use a relatively high dose of a conventional cancer therapeutic drug or to simultaneously use a combination of these drugs in the hope that as many of the cancer cells as possible are destroyed before they have developed with MDR. The drawback is that this will cause enormous side effects to the patient and deter drug compliance. Very often, even with full drug compliance further remission may still occur. In some cases, cancer cells are found to be MDR even before any treatment has began.

Ling et al (Ling, V., Cancer chemother. Pharmacol., 40 (Suppl.): S3-S8, 1997) identified a 170 kD membrane P-glycoprotein (P-gp), which was subsequently found to mediate ATP-dependent efflux of certain cancer therapeutics from multi-drug resistant (MDR) tumor cells. These cancer therapeutics include anthracyclines, Vinca alkaloids, epipodophyllotoxins, and taxanes. P-gp is resident in plasma membranes and functions as an efflux transporter of natural-product lipophilic xenobiotics.

Studies carried out over the last several years have demonstrated that intrinsic and acquired expression of P-gp plays a major role in clinical MDR. Tumor types that frequently express P-gp in the absence of exposure to chemotherapy include colorectal, renal cell, hepatocellular, and adrenocortical cancers, as well as chronic leukemia.

Several additional tumor types express P-gp at diagnosis in approximately 10-50% of cases. Examples include breast carcinoma, acute myelogenous leukemia, and ovarian carcinoma (Nussler, V., Pelka-Fleischer, R., Zwierzina, H., Nerl, C., Beckert, B., Gieseler, F., Diem, H., Ledderose, G., Gullis, E., Sauer, H., and Wilmanns, W., Leukemia, 10 (Suppl. 3): S23-S31, 1996). P-gp expression at diagnosis in these tumor types can play a significant role in treatment outcome. For example, patients with breast carcinomas expressing P-gp are three times more likely to fail to respond to chemotherapy than patients whose tumors are P-gp negative.

Chemotherapeutic agents such as doxorubicin can select for mutations leading to increased expression of P-gp and a P-gp inhibitor has been found to suppress activation of MDR1 gene expression and decrease the mutation rate for resistance to doxorubicin (Chen, G. Jaffrezou, J. P., Fleming, W. H., Duran, G. E., and Sikic, B. I., Cancer Res., 54: 4980-4987, 1994).

Reversal of MDR is one of the major goals in cancer chemotherapy. Potent reversal agents for P-gp have been identified and clinical trials using these agents are ongoing. The first attempt to reverse P-gp-mediated MDR is usually by calcium channel blockers such as verapamil and cyclosporin A (Hindenburg, A. A., Baker, M. A., Gleyzer, E., Stewart, V. J. , Case, N., and Taub, R. N., Cancer Res., 47: 1421-1425, 1987). Although having some efficacy, these agents are relatively weak P-gp inhibitors (EC₅₀s, 2-10 μM), are often substrates for P-gp, and exhibit dose-limiting side effects that severely restrict their clinical utility. To address these problems, there has been considerable interest in identifying second-generation P-gp inhibitors, which do not elicit significant toxicity at doses required for P-gp inhibition. Common dose-limiting toxicities for these types of compounds are ataxia and hyperbilirubinemia, which are reversible upon cessation of drug treatment.

Apoptosis was initially described by its morphological characteristics, including cell shrinkage, membrane blebbing, chromatin condensation and nuclear fragmentation. Since apoptotic programs can be manipulated to produce massive changes in cell death, the gene and proteins controlling apoptosis are potential drug targets. Many empirically derived cytotoxic drugs already may target apoptosis, albeit indirectly and non-exclusively. However, they are also mutagenic and toxic to normal tissues. In contrast, agents that directly induce apoptosis may provide less opportunity for acquired drug resistance, decrease mutagenesis and reduce toxicity.

Historically, plants have been used in the treatment of cancer. For instance, many Chinese medicinal herbs have been used to improve the general conditions of the cancer patients, but they have not been known to have been used in the specific eradication of cancer cells generally, or MDR cancer cells in particular. In any event, nature has provided many plant-derived anti-cancer drugs, such as vinblastine, irinotecan, topotecan, etoposide and paclitaxel.

The present invention is directed to a set of related chemical compounds which may be used as therapeutics for prevention and/or treatment against cancer cells, and in particular MDR cancer cells, when used alone or in combination with other therapeutics for cancer.

### SUMMARY OF THE INVENTION

According to a first aspect of the present invention, there is provided chemical compounds of the general formula as shown in FIG. 1. Each of the R1 and R2 groups may be a saturated or unsaturated acid. The saturated or unsaturated acid may be an acid having two to five carbons. In particular, the R1 or R2 group may be selected from a group including —OCOCH₃ and —OCOCCH₃═CHCH₃. In particular, the chemical compounds may comprise cis-3′-angeloyl-4′-acetoxy-khellactone. The chemical compounds may also comprise cis-3′-acetoxy-4′-angeloyl-khellactone, trans-3′-angeloyl-4′-acetoxy-khellactone, or trans-3′-acetoxy-4′-angeloyl-khellactone.

One main difference between these chemical compounds resides in the relative location (i.e. cis or trans location) of the saturated or unsaturated acid within the relevant compound, this however should not affect their therapeutic activities against cancer cells.

According to a second aspect of the present invention, there is provided chemical compounds as described above wherein the chemical compounds are isolated from a natural source. In particular, the natural source is the plant of Peucedanum praeruptorum Dunn (Umbelliferae).

According to a third aspect of the present invention, there is provided chemical compounds as described above wherein the chemical compounds are produced by synthesis, such as conventional methods of chemical synthesis.

According to a fourth aspect of the present invention, there is provided use of chemical compounds as described above for the manufacture of a medicament for the treatment of cancer. The medicament may be adapted to treat cancer caused by multi-drug resistant (MDR) cancer cells in particular. The medicament may be adapted to cause apoptosis in cancer cells.

According to a fifth aspect of the present invention, there is provided a composition comprising at least one of chemical compounds as described above and a chemical substance selected from a group including doxorubicin, anthracycline, Vinca alkaloid, epipodophyllotoxin and taxane.

According to a sixth aspect of the present invention, there is provided use of a composition as described above for the manufacture of a medicament for the treatment of cancer.

According to a seventh aspect of the present invention, there is provided a method of isolating one or more chemical compounds as described above from the plant known as Peucedanum praeruptorum Dunn (Umbelliferae). The method may comprise the steps of (a) drying the plant, (b) treating the plant with 30 to 1000 wt % of an alcohol for at least one hour to produce an extract therefrom; (c) filtering the extract by a suitable filter paper; (d) removing the alcohol from the filtered extract to produce a first residue; (e) purifying the first residue by extraction with an organic solvent, e.g. chloroform, to produce a second residue; (f) purifying the second residue by column chromatography; and (g) producing crystals comprising at least one of the chemical compounds by re-crystallizing the purified second residue. The method may further comprise a step (h) of powdering the plant, for example after step (a). Preferably, the alcohol may be ethanol although other alcohols such as methanol or propanol may also work. The filter paper may be a No. 2 filter paper. The set of chemical compounds isolated from the plant may comprise (+)-cis-3′-angeloyl-4′-acetoxy-khellactone and/or (−)-cis-3′-angeloyl-4′-acetoxy-khellactone.

### BRIEF DESCRIPTION OF THE DRAWINGS

The present invention is described, by way of examples and experiments only, with reference to the following drawings in which:

FIG. 1 shows a general structural formula of a set of chemical compounds having a pyranocoumarin group;

FIGS. 2 and 3 show structural formulae of two of the chemical compounds shown in FIG. 1;

FIG. 4 illustrates the structure of the chemical compounds shown in FIGS. 2 and 3 by NMR-COSY technique;

FIG. 5 illustrates the structure of the chemical compounds shown in FIGS. 2 and 3 by NMR-NOESY technique;

FIGS. 6 & 7 show the three-dimensional structure of the conformers of the chemical compound shown in FIG. 2;

FIGS. 8 and 9 are two agarose gel images showing DNA ladders of KB-3-1 cells and KB-VI cells in an experiment;

FIG. 10 is an agarose gel image showing DNA ladders of KB-3-1 and KB-V1 cells in another experiment;

FIGS. 11 and 12 are graphs showing the effectiveness and synergistic effects of various combinations of drugs on KB-3-1 and KB-V1 cells respectively in an experiment;

FIGS. 13 and 14 are graphs showing the effectiveness and synergistic effects of various combinations of drugs on KB-3-1 and KB-V1 cells respectively in another experiment;

FIG. 15 is a gel image showing effects of a drug on KB-3-1 and KB-V1 cells respectively; and

FIGS. 16 and 17 are graphs showing effects of a drug on the accumulation of doxorubicin in KB-3-1 and KB-V1 cells respectively.

### DETAILED DESCRIPTION OF THE PRESENT INVENTION

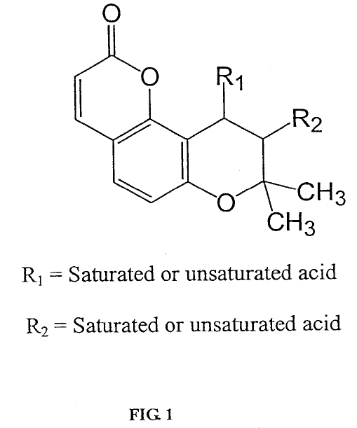

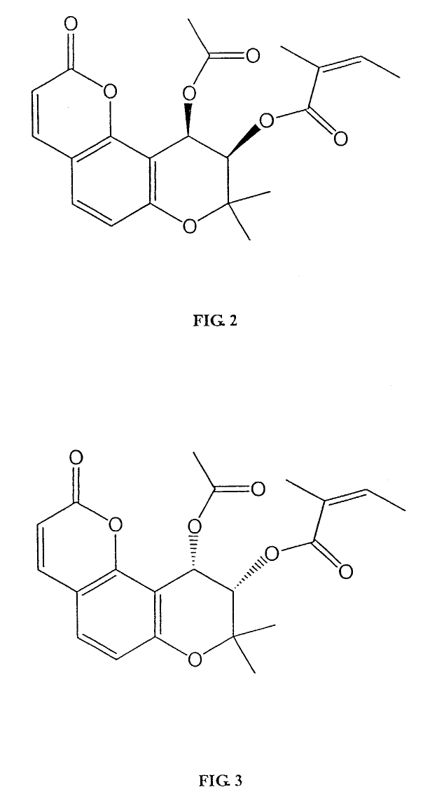

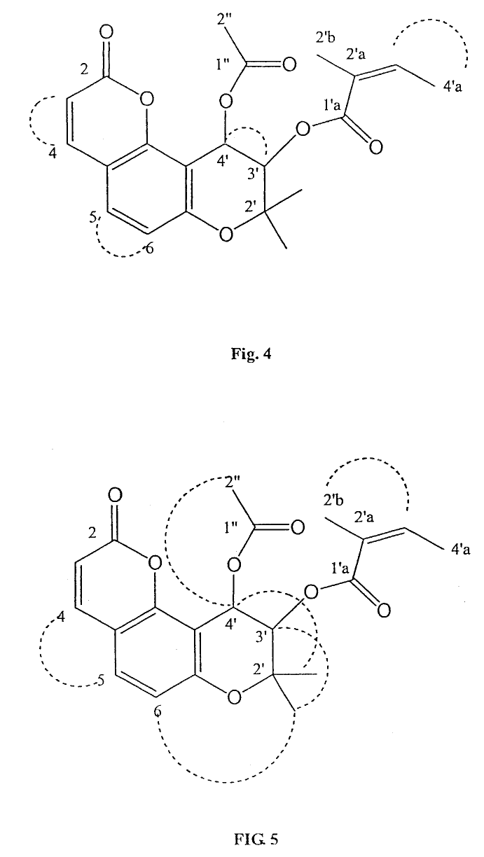

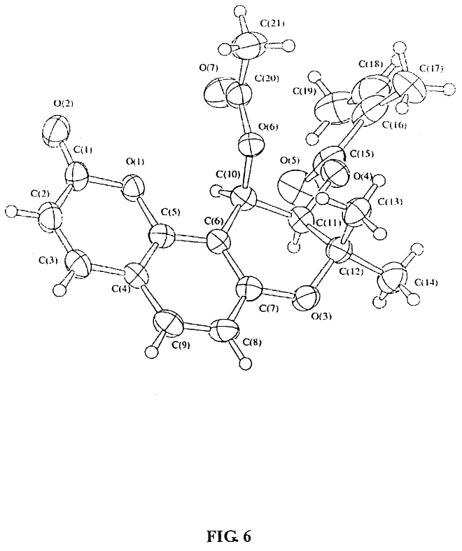

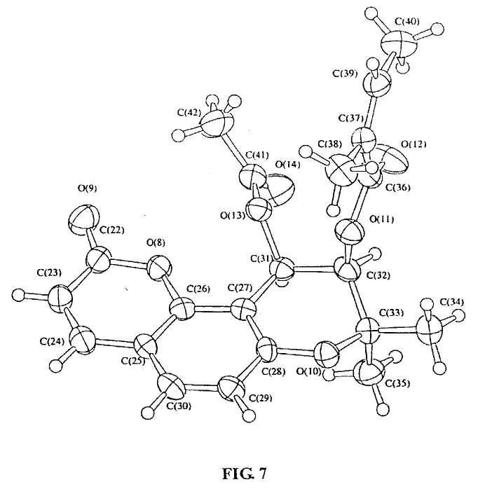

The present invention is generally concerned with a set of closely related chemical compounds or their variations (or derivatives) having therapeutic activity in treating cancer. Each of the chemical compounds comprises a pyranocoumarin group and these chemical compounds share the general structural formula which is represented in FIG. 1. In particular, the present invention is also concerned with the isolation and identification of at least two of the related chemical compounds having activity in suppressing and/or causing apoptosis in cancer cells, and the two chemical compounds are “(+) -cis-3′-angeloyl-4′-acetoxy-khellactone” and “(−)-cis-3′-angeloyl-4′-acetoxy-khellactone”, both of which are referred as “APC” hereinafter. During the course of the present invention, APC has been firstly successfully isolated from the Chinese medicinal herb called “Bai-Hua Qian-Hu” which is actually the root of Peucedanum praeruptorum Dunn (Umbelliferae). Different structural formulae of APC are illustrated in FIGS. 2 & 3. FIG. 4 shows the correlation—interaction of adjacent protons at various locations in the molecule—observed in the ¹H-¹H correlated spectroscopy (COSY) NMR spectra. FIG. 5 shows the correlation—interaction of intra-molecular protons through space—observed in the nuclear overhauser effect spectroscopy (NOESY) NMR spectra. FIGS. 6 & 7 illustrates the three-dimensional structures of the conformers of the chemical compound shown in FIG. 2. The structure of APC was elucidated by X-ray structural analysis, mass spectrometry and NMR spectroscopy including extensive two-dimensional NMR studies.

As indicated above, the Chinese herb Bai-Hua Qian-Hu is the dry root of Peucedanum praeruptorum Dunn. This herb is officially listed in the Chinese Pharmacopoeia and it has been reported that it may be used as expectorant and mucolytic. It has been found that Bai-Hua Qian-Hu comprises APC (which is a member within the set of related chemical compounds discussed above) which comprises a number of angular-type, non-glycosidic pyranocoumarins. However, the variations of APC within the set of chemical compounds may or may not be of angular-type. These variations also possess similar therapeutic activity in treating cancer but with lower effectiveness. The characteristics of APC in particular are explained in further details as follows.

Furthermore, the chemical compounds and compositions of the present invention generally include a compound of the general formula as shown in FIG. 1 or a physiologically acceptable salts or prodrugs of the compound. Each of the R1 and R2 groups may be a saturated or unsaturated acid. The saturated or unsaturated acid may be an acid having two to five carbons. In particular, the R1 or R2 group may be selected from a group including —OCOCH₃ and —OCOCCH₃═CHCH₃. In particular, the chemical compounds may comprise cis-3′-angeloyl-4′-acetoxy-khellactone. The chemical compounds may also comprise cis-3′-acetoxy-4′-angeloyl-khellactone, trans-3′-angeloyl-4′-acetoxy-khellactone, or trans-3′-acetoxy-4′-angeloyl-khellactone.

As previously suggested the compounds of the present invention may be provided as physiologically acceptable salts wherein the claimed compound may form the negatively or the positively charged species. Examples of salts in which the compound forms the positively charged moiety include, without limitation, quaternary ammonium, salts such as the hydrochloride, sulfate, carbonate, lactate, tartrate, maleate, succinate, etc. formed by the reaction of an amino group with the appropriate acid. Salts in which the compound forms the negatively charged species include, without limitation, the sodium, potassium, calcium and magnesium salts formed by the reaction of a carboxyic acid group in the molecule with the appropriate base (e.g. sodium hydroxide (NaOH), potassium hydroxide (KOH), Calcium hydroxide (Ca(OH).sub.2), etc.).

Additionally, a “prodrug” refers to an agent which is converted into the parent drug in vivo. Prodrugs are often useful because, in some situations, they may be easier to administer than the parent drug. They may, for instance, be bioavailable by oral administration whereas the parent drug is not. The prodrug may also have improved solubility in pharmacological compositions over the parent drug. An example, without limitation, of a prodrug would be a compound of the present invention wherein it is administered as an ester (the “prodrug”) to facilitate transmittal across a cell membrane where water solubility is not beneficial, but then it is metabolically hydrolyzed to the carboxyic acid once inside the cell where water solubility is beneficial.

Pharmacological compositions of the compounds and the physiologically acceptable salts and prodrugs thereof are preferred embodiments of this invention. Pharmacological compositions of the present invention may be manufactured by processes well known in the art; e.g., by means of conventional mixing, dissolving, granulating, dragee-making, levigating, emulsifying, encapsulating, entrapping or lyophilizing processes.

Pharmacological compositions for use in accordance with the present invention thus may be formulated in conventional manner using one or more physiologically acceptable carriers comprising excipients, adjuvants, diluents and auxiliaries which facilitate processing of the active compounds into preparations which can be used pharmaceutically. Proper formulation is dependent upon the route of administration chosen.

For injection, the compounds of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmacological preparations for oral use can be made with the use of a solid excipient, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmacological compositions which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmacological compositions for parenteral administration include aqueous solutions of the active compounds in water soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. optionally, the suspension may also contain suitable stabilizers or agents which increase the solubility of the compounds to allow for the preparation of highly concentrated solutions.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. The pharmacological compositions herein also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

As previously mentioned, the form in which the composition will be administered (e.g., powder, table, capsule, solution, emulsion) will depend on the route by which it is administered. However, the quantity of the composition to be administered will be determined on an individual basis, and will be based at least in part on consideration of the severity of condition. of the patient, the patient's overall health, the patient's weight, the time available before other treatment and the means of administration (e.g. a larger amount may be administered for oral compositions than for systemic compositions). In general, a single dose will normally contain approximately 0.01 mg to 500 mg of the compounds or compositions of the present invention per kilogram of body weight, preferably 1 mg to 250 mg per kilogram of body weight, more preferably 2 mg to 20 mg per kilogram of body weight.

### Experiment 1

### Chemical Studies

The infrared spectrum of APC exhibits the presence of α-pyrone ring (1740.86 cm⁻¹), the aromatic ring (1654.23, 1607.50, 1492.08 cm-⁻¹) and aryl ester groups (1284.79, 1235.80, 1147.73, 1115.20 cm⁻¹). The mass spectrum of APC shows a molecular peak at m/z 409 [M+Na]⁺ (100), 349.2 (13), 327.2 (20), 284.2 (13), 227.2 (46), 198.9 (7), 83 (43), 55 (78).

(The ¹H-NMR spectrum in the aromatic proton region of APC contains two pairs of doublets at δ=6.24 and 7.60 (each 1H, J=9,53 Hz) and δ=7.36 and 6.80 (each 1H, J=8.51 Hz), which are in agreement with H-3 and H-4 signals of the α-pyrone ring system and a significant o-coupling signal due to H-5 and H-6 on the angular coumarin ring. A pair of doublets at δ=5.41 and 6.60 ppm (each 1H, d, J=4.99 Hz) is assigned to the methine protons at C-3′ and C-4′ of cis-khellactone which showed a characteristic splitting pattern (J=4.99 Hz). A quartet quartet coupling at δ6.13 (1H, qq, J=7.33, 1.47 Hz) to a doublet quartet coupling at δ=1.96 (3H, dd, J=7.33, 1.47 Hz) were assigned to H-3′a and H-4′a respectively. The signal at δ=2.11 ppm (3H, s) is due to an acetyl group at H-2″ and the quintet signal at δ=1.87 ppm (3H, qu, J=1.47 Hz) results from the H-2′b at 2-methyl-butyrate moiety. Two close singlets at δ=1.47 & 1.43 ppm (Δ=0.04 ppm), due to the 2′-gem-(Me)₂ groups of a dihydropyran ring, indicate a cis-configuration at 3′-H and 4′-H. The assignments of ¹H NMR resonance of APC were determined through analysis of its ¹H-¹H COSY and NOESY data as shown in FIGS. 4 & 5.

Re-crystallization of APC from ethanol-n-hexane yielded a crystal of sufficient quality of APC for X-ray structural analysis, which on completion led to the solution of two enantiomers in the dihydropyran ring of APC as shown in FIGS. 2 and 3, if which each chemical also led to the solution of two conformers as shown in FIGS. 6 & 7 and with the atomic positional and isotropic displacement parameters shown in Table 1.

Consequently, the structure of APC in FIGS. 2 and 3 are “(+) -cis-3′-angeloyl-4′-acetoxy-khellactone” and “(−)-cis-3′-angeloyl-4′-acetoxy-khellactone”. The variations of APC (i.e. the other members of the set of chemical compounds) include but not limited to “(+)-trans-3′-angeloyl-4′-acetoxy-khellactone” and “(−)-trans-3′-angeloyl-4′-acetoxy-khellactone”.

Two structures of APC were elucidated by X-ray structural analysis, mass spectrometry and NMR spectroscopy including extensive 2D NMR studies as C₂₁H₂₂O₇, Mr=386.40 (FIGS. 2 and 3).

### Experiment 2

### Biological Effects

The level of a lethal dose (LD) of a chemical compound at which half of cells is destroyed at such dose is known as LD₅₀. KB-V1 is known to be a MDR cancer cells and KB-3-1 is a drug sensitive cancer cell which is not MDR and is sensitive to conventional cancer therapeutic drugs. This experiment was aimed to ascertain the cytotoxicity of APC and doxorubicin on drug sensitive cancer cells such as KB-3-1.

Assays were carried out in triplicate against human carcinoma KB-3-1 cancer cell line. In particular, Sulfo-Rhodamine B (SRB) assay (Skehan P.; Storeng R.; Scudiero D.; Monks A.; McMahon J.; Vistica D; Warren J T.; Bokesch H.; Kenney S.; Boyd M R.; J Natl Cancer Inst 1990 82:1107-1112) was used to determine the cytotoxicity of APC and doxorubicin. The treatment of KB-3-1 cancer cells lasted for 72 hours and the cell number in each sample was estimated by correlating to optical density (OD) at 515 nm. In the assays, doxorubicin was used as the positive cytotoxic control drug. The median dose value was determined from plots of median effects and was equivalent to LD₅₀ (Skehan P.; Storeng R.; Scudiero D.; Monks A.; McMahon J.; Vistica D; Warren J T.; Bokesch H.; Kenney S.; Boyd M R.; J Natl Cancer Inst 1990 82:1107-1112).

Results and Discussion:

It was shown that after treatment of the KB-3-1 cells by APC, LD₅₀ was achieved. In particular, it was found that the LD₅₀s of APC and doxorubicin were 41.9153±2.8016 μM and 0.0639±0.0106 μM respectively. This means a much higher dose of APC (i.e. 41.9153±2.8016 μM) is required to produce the same killing effect of doxorubicin. The ratio of the two values of LD₅₀ is about 650, and this means that APC is about 650 times less toxic than doxorubicin.

The above data indicates that while APC may be used in suppressing and treating cancer cells, however, APC when used alone is not as effective as doxorubicin on drug sensitive cancer cells.

### Experiment 3

### Activity of APC Against Drug Sensitive Cancer Cells

Experiment 2 above shows that APC does, to an extent, have cytotoxic activity against the cancer cells. Two possible causes of the death of the cancer cells are “sudden cell death” and “programmed cell death”. In simple terms, sudden cell death means that the cancer cells are poisoned and caused to die or rupture right away once treated with the drug. For sudden cell death, there is usually little or no control on how and when the cell death would occur. On the other hand, programmed cell death means the cancer cells will go through certain physiological processes in a controlled and/or orderly manner before cell death occurs. This experiment was aimed to demonstrate the effect of different concentrations of APC on normal cancer cells (or more commonly referred to “drug sensitive cancer cells”) which are sensitive to conventional cancer therapeutic drugs such as doxorubicin. Exactly how the death of cancer cells occurs will be dealt with briefly in later experiments and discussions.

In this experiment, three groups of KB-3-1 cells were treated for 24 hours at different concentrations (17.26 μM, 34.52 μM & 172.56 μM) of APC. A control group of KB-3-1 cells was treated as negative control for the same amount of time. Approximately 5×10⁶ cells from each of the four groups were washed in phosphate buffered saline (PBS) and their genomic DNA was extracted using Apoptotic DNA Ladder Kit-Best. Nr. 1 835 246 (Roche) according to the manufacturer's manual. The extracted DNA was analyzed by electrophoresis in 2% agarose gel containing 0.1% ethidium bromide in tris galacial acetic acid (TAE) buffer for 30 minutes at 99v. DNA bands of the gel were observed and recorded by Multi-Imager (Bio-Rad, California, USA).

Results and Discussion:

An image of the gel is shown in FIG. 8. Five lanes are shown with the first lane from the left being the marker lane and the second lane from the left being the control lane. In the control lane, the band shown therein is fairly concentrated and bright which means that the DNA therein originated from the control sample of cells generally had remained fairly intact and uncut. The genomic DNA originated from the control group of KB-3-1 cells were not treated with any drug. In the right most lane, it is shown that the band therein is relatively spread out along the lane and faint. The DNA sample of this lane originated from a test group of KB-3-1 cells which had been treated with a relatively high concentration of 172.6 μM of APC. This means that the DNA in those cells had been cut into smaller fragments. This illustrates that the DNA of the KB-3-1 cells had been digested after they had been treated with APC. It is indicative that apoptosis had been caused on these cells and their DNA had been fragmented.

It is shown that when administered with 172.6 μM of APC, there is a significantly more apoptosis of the KB-3-1 cells indicated by the DNA fragments in the right most column. It is thus shown that APC is a mild cancer growth inhibitory drug with prominent apoptotic activity at a relatively high concentration against drug sensitive cancer cells such as KB-3-1 type cells.

### Experiment 4

### Isolation of APC from the Chinese Herb

The isolation of the set of related chemical compounds and in particular the two enantiomers of APC involved the use of conventional apparatus to detect various parameters. Melting points were determined using Electrothermal 8100 melting point apparatus and were uncorrected. Optical rotations were measured on a Jasco DIP-370 Digital Polarimeter. Fortier Transform Infrared (FTIR) spectra was recorded on a Perkin-Elmer 1600 spectrophotometer, and UV spectra was taken on a Shimadzu UV-3100 spectrophotometer. NMR spectra was recorded in CDCl₃ solution on a Varian NMR-300 MHz spectrometer using tetramethylsilane (TMS) as internal standard. Silica gel 60 (200-300 mesh) was used for column chromatography. C18 Rocket Silicon Column and PE Series 200 Micro Pump were used for HPLC. 60% methanol was used as solvent system and UV detector was set at 320nm. Liquid chromatography/mass spectroscopy/mass spectroscopy (LC/MS/MS) was recorded on a PE SCIEX API 365 LC/MS/MS System.

The Chinese herb “Bai-Hua Qian-Hu” (Peucedanum praeruptorum Dunn) used in the experiment was purchased from the Anhui Province of China. The herb is generally purchasable in many Chinese herbal stores in Asia including China and Hong Kong.

The procedure of isolating APC from Bai-Hua Qian-Hu (Peucedanum praeruptorum Dunn) firstly involved removing and/or extracting the water content from the herb. This may be done by drying the herb using conventional drying method. The dried herb may then be powdered and then treated with 50% cool ethanol for about 72 hours although treating the (dried and/or powered) herb with 30% to 100% of a suitable alcohol (e.g. methanol or propanol) for not less than 1 hour will deliver similar result but with lower efficiency. Impurities are then removed from the solvent (i.e. ethanol) by filtering it through a filter paper such as a No. 2 filter paper. Ethanol was then removed from the filtered solvent by evaporation using a rotary evaporator. Residue left after evaporation was then treated with chloroform. The chloroform was then removed by evaporation using a rotary evaporator leaving behind a crude substance which was then purified by column chromatography on a silica gel using methanol-Diethyl ether (1:5) as the solvent, although other types of purification method may also be used. Repeated re-crystallization of the purified crude substance from ethanol gave crystals of APC with its molecular structure having a pyranocoumarin group and a coumarin group.

Using the above method, APC would normally be obtained as white powder (EtOH) and was measured to have the following properties: [mp 153-154° C.; [α]_{D}²⁰ 0° (c0.03, CHCl₃); UV (EtOH) λₘₐₓ (log ε) at 25 μg/ml 324 (0.984), 255(0.24), 210 (1.472); IR υₘₐₓ^{KBr} cm⁻¹ 1740.86, 1654, 1608, 1492, 1285, 1236, 1148, 1115, 847; ¹H-NMR (CDCl₃, 300 MHz) δ7.60 (1H, d, J=9.53 Hz, H-4), 7.36 (1H, d, J=8.51 Hz, H-5), 6.80 (1H, d, J=8.51 Hz, H-6), 6.60 (1H, d, J=4.99 Hz, H-4′), 6.24 (1H, d, J=9.53 Hz, H-3), 6.13 (1H, qq, J=7.33, 1.47 Hz, H-3′a), 5.41 (1H, d, J=4.99 Hz, H-3′), 2.11(3H, s, H-2″), 1.96 (3H, dq, J=7.33, 1.47 Hz, H-4′a), 1.87 (3H, qu, J=1.47 Hz, H-2′b), 1.47 & 1.43 (3H, s, gem-(Me)₂); EIMS m/z 409 [M+Na]⁺ (100), 349.2 (13), 327.2 (20), 284.2 (13), 227.2 (46), 198.9 (7), 83 (43), 55 (78); CHN elemental analysis, anal. C 65.00%, H 6.08%, calcd for C₂₁H₂₂O₇, C 65.28%, H 5.74%.

### Experiment 5

### Crystal Data of APC

The APC crystals were further recrystalized from ethanol/n-hexane. The physical data of the crystal is as follows. C₂₁H₂₂O₇, Mr=386.40; monoclinic, space group P2₁/c(#14), a=16.970(1)Å, b=12.5520(7) Å, c=18.671(1) Å, β=97.94(1) Å, V=3938.9(4) Å³. D_{c}=1.303 g/cm³; F₀₀₀=1632.00; μ(MoKα)=0.98 cm⁻¹; specimen: 0.22×0.11×0.07 mm; nᵥ=505; |Δρmax|=0.14 e Å⁻³.

### Experiment 6

### Atomic Positional and Isotropic Displacement Parameters

In this experiment, the parameters of the atomic positional and isotropic displacement of APC were obtained, which are shown in Table 1.

In addition of the above experiments, it is demonstrated in the following experiments that in Peucedanum praeruptorum Dunn (Umbelliferae), and in particular in the root and extracts of Peucedanum praeruptorum Dunn (Umbelliferae), natural APC and its variations, and synthetic APC and its variations may be used in treating cancer. In the past, Bai-Hua Qian-Hu has been known to be useful in treating common cough and resolving phlegm but there has been no disclosure or sufficient data showing that it may be useful in treating cancer.

In the following experiments, tests were conducted using the human cancer cell lines KB-3-1 and KB-V1. The MDR cancer cells KB-V1 were selected from drug sensitive KB-3-1 cells treated by vinblastine. The cell lines (American Type Culture Collection, MD, USA) were routinely grown in MEM (Gibco, MD, USA) containing 10% fetal bovine serum (FBS) and 1% antibiotic solution (Gibco, MD, USA), and 300 ng/ml of vinblastine for maintenance of drug resistance for KB-V1 cells. Cultures of the cells were maintained in a humidified 5% CO₂ incubator. Vinblastine, doxorubicin, and verapamil were purchased from Sigma Chemical Co. The APC used in the experiment was isolated and characterized from Peucedanum praeruptorum Dunn using the method described above in Experiment 4. The APC used had a purity of over 98% as judged by NMR and HPLC.

### Experiment 7

### Cell Proliferation Assays

Cell proliferation (or LD₅₀) of the APC and doxorubicin alone was determined from 3-day dose-response curves carried out in triplicate in 96-well plates essentially as described in Sulfo-Rhodamine B (SRB) cytotoxicity assay (Skehan P.; Storeng R.; Scudiero D.; Monks A.; McMahon J.; Vistica D; Warren J T.; Bokesch H.; Kenney S.; Boyd M R.; J Natl Cancer Inst 1990 82:1107-1112) and Median-effect equation (see Appendix towards the end of the description for explanation). The color intensity of SRB, positively correlated to number of cell, was estimated by correlating to OD at 515 nm. The concentration of DMSO and ethanol in the drug solvent of APC was 20 wt % and 80 wt % respectively. The concentration of DMSO in each culture sample was ≦0.1%.

Results and Discussion:

Solvent controls were run with the assays and it was found that no cytotoxic effects were produced in the controls.

Table 2a summarizes the results of the experiment. In particular, the LD₅₀s for the doxorubicin and APC were determined by SRB cytotoxicity assays. The LD₅₀s ratio of MDR KB-V1 cells to drug sensitive KB-3-1 cells indicates the degree of refractory or resistance of MDR cells towards a particular drug. The results were obtained in three independent tests.

It is shown that doxorubicin, when compared with APC, is more effective in treating drug-sensitive KB-3-1 cells in that a lower dose of doxorubicin is required to achieve LD₅₀. Since KB-3-1 cells are non-MDR, this indicates that APC alone is less effective in treating drug sensitive (or non-MDR) cancer cells.

However, it is shown that APC, when compared with doxorubicin, is more effective in treating KB-V1 cells. Since KB-V1 cancer cells are MDR, this indicates that APC is more effective in treating MDR cancer cells. In other words, APC has preferential killing against MDR cancer cells.

### Experiment 8

### Apoptosis Studies I

This experiment was a continuation of Experiment 7. Apoptotic studies by DNA ladder was carried out for 24 hours with drug treatment at different concentrations (17.26 μM, 34.52 μM & 172.56 μM) of APC. A total of eight groups of cells were used. 5×10⁶ cells were washed in PBS. Their genomic DNA was extracted using Apoptotic DNA Ladder Kit-Best. Nr. 1 835 246 (Roche) according to Manufacturer's manual. DNA was analyzed by electrophoresis in 2% agarose gel containing 0.1% ethidium bromide in TAE buffer (99v, 30 min). DNA bands were observed and recorded by Multi-Imager (Bio-Rad, California, USA).

Results and Discussion:

Referring to Table 2a, Sulfo-Rhodamine B (SRB) cytotoxicity assay and Median-effect equation indicated that doxorubicin has LD₅₀s of 0.0639±0.0106 μM and 3.0510±0.2846 μM for drug-sensitive KB-3-1 and MDR KB-V1 respectively. However, APC has abnormal LD₅₀s of 41.9153±2.8016 μM and 17.2656±8.2441 μM for drug-sensitive KB-3-1 and MDR KB-V1 respectively. The LD₅₀ ratio of APC on MDR KB-V1 cells to drug-sensitive KB-3-1 cells suggests that MDR KB-V1 is about 2.4 times more sensitive to APC than drug-sensitive KB-3-1. The results indicate that APC has a preferential killing activity against MDR cancer cells.

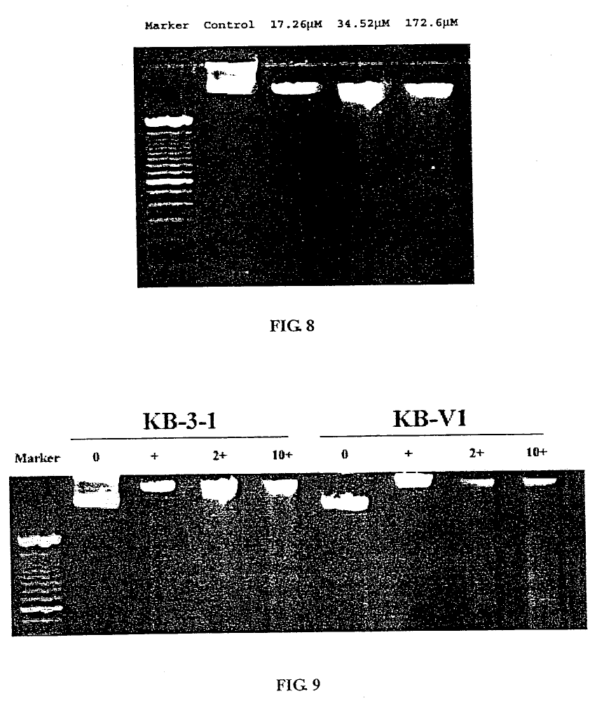

FIG. 9 shows the DNA ladders from the experiment. The agarose gel image has nine lanes. Lane no. 1 is the marker lane. Lane nos. 2 to 5 and lane nos. 6 to 9 are DNA ladders showing DNA samples obtained from the KB-3-1 and KB-V1 cells respectively after drug treatment.

In lane nos. 2 and 6, the DNA samples were resulted from KB-3-1 and KB-V1 cells respectively not having treated with any drug. The bands shown therein are relatively condensed and arranged at the upper part of the lanes indicating that the DNA was not cut and this suggests that the cells thereof were little affected.

In lane nos. 3 and 7, the KB-3-1 and KB-V1 cells corresponding to these lanes were treated with 1+ (i.e. 17.26 μM) APC. The band in lane no. 3 is still relatively condensed compared to those in lane nos. 2 and 7 indicating that the DNA from the KB-3-1 cells was little affected. However, the fluorescence of the band in lane no. 7 is relatively slightly more spread out indicting that the DNA was slightly cut and this suggests that at least some of the KB-V1 cells thereof were killed.

In lane nos. 5 and 9, the KB-3-1 and KB-V1 cells corresponding to these lanes were treated with 10+ (i.e. 172.6 μM) APC. The band in lane no. 9 is most spread out and least condensed among lane nos. 2 to 9. This indicates that DNA from the KB-V1 sample was cut in larger extent and this suggests that relatively large portion of the KB-V1 cells thereof were killed. It can thus be concluded that APC generally is more effective in causing apoptosis in and killing MDR cancer cells.

The apoptosis study above suggests that the preferential killing by APC correlates to some unknown cytostatic apoptotic pathways. The DNA ladders suggest that the apoptotic induction effects of APC are dose-dependent and MDR KB-V1 cells were more susceptible than the drug-sensitive KB-3-1 cells at the same concentration of APC. The results illustrate that APC has a dose-dependent preferential apoptotic induction effect against MDR cells.

### Experiment 9

### Apoptosis Studies II

Similar to Experiments 7 & 8, this experiment was sought to study apoptosis on KB-3-1 and KB-V1 cells. However, instead of using APC and doxorubicin, the crude extract of Bai-Hua Qian-Hu comprising APC, and doxorubicin were used instead.

Results and Discussion:

Similar to Table 2a, Table 2b illustrates that doxorubicin, when compared with the crude extract, is more effective in treating drug-sensitive KB-3-l cells. The crude extract is more effective in treating KB-V1 cells than KB-3-1 cells. Since KB-V1 cancer cells are MDR, this indicates that the crude extract having APC is more effective in treating MDR cancer cells. In other words, crude extract of the plant also has preferential killing effect against MDR cancer cells.

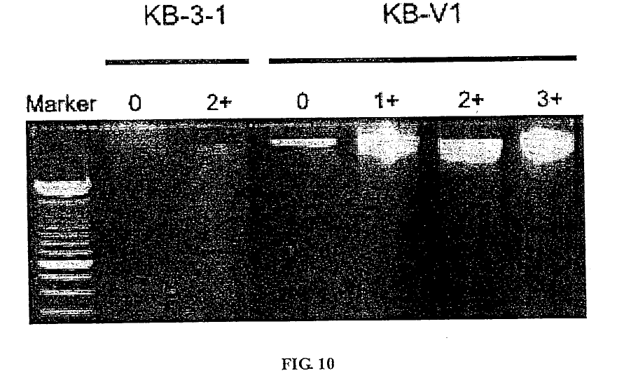

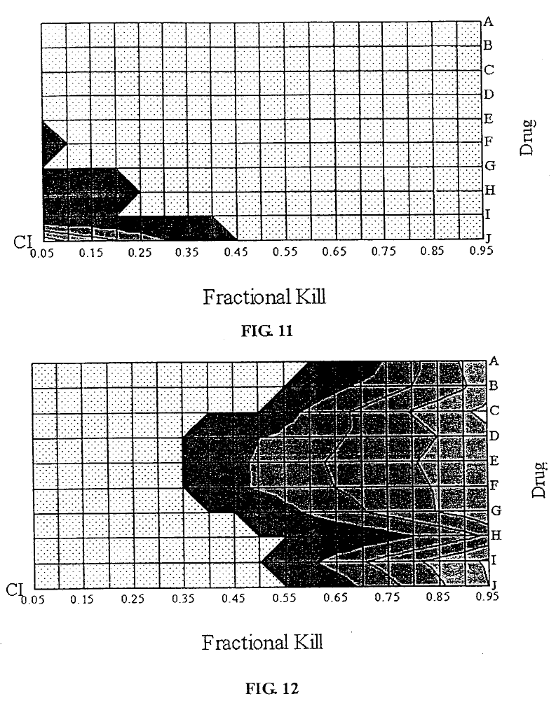

FIG. 10 shows the DNA ladders from the experiment. It is shown that a relatively low dose of the crude extract was sufficient to achieve a LD₅₀ on the KB-V1 cells than on the KB-3-1 cells indicating that MDR cancer cells such as KB-V1 cells are more susceptible to apoptosis on treatment with the crude extract. The crude extract comprised APC.

### Experiment 10

### Effects of the Drug Combination of APC and Doxorubicin

In this experiment, the CI-isobologram by Chou and Talalay was used to determine the Combination Index (CI) of APC and doxorubicin. In order to understand the synergistic effect of APC with a conventional cancer drug, a number of combinations of drug concentrations were used in the combination studies. Tables 3a to 4b show the two groups of drug combinations A to J for KB-3-1 and KB-V1 respectively. A mutually non-exclusive equation was used to determine the CIs, and the equation is illustrated and explained in the Appendix section below. CI>1, CI=1, and CI<1 indicate antagonism, additive effect, or synergism, respectively. The explanation of the determination of combination indices (CI) is provided in the Appendix section towards the end of the description.

Results and Discussion:

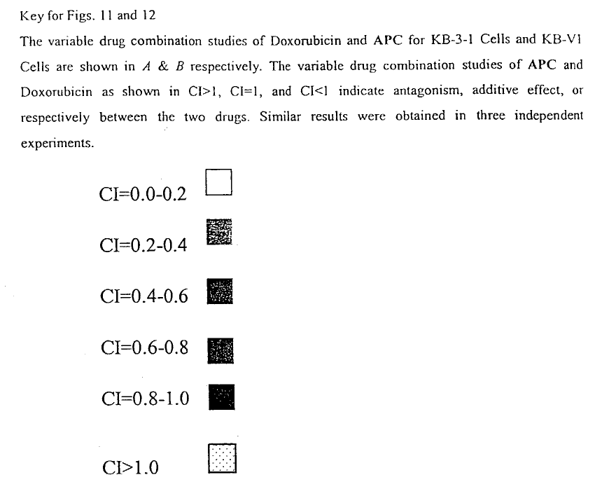

FIGS. 11 and 12 are graphs showing the effect of different combinations of APC and doxorubicin on KB-3-1 and KB-V1 cells respectively. The graphs are to be read in conjunction with the key following the figures and Tables 3a to 4b. A low CI value means that the drug composition has high synergism therein. A CI value of “0” means that the drug combination has the highest synergism. A high fractional kill means that the drug combination can kill a relatively high percentage of the cancer cells. A CI value of “0” coinciding with the high fractional kill value of “1” means that the drug combination not only have maximum synergism therein but is also effective in producing a complete kill (i.e. 100%) of the cancer cells. It is also to be noted that a CI value of >1 means that there is antagonism in the drug combination.

Referring specifically to FIG. 11, it is shown that none of the area in the graph shows an area having a CI value of “0” (i.e. a complete white area, please refer to the Key for reference) indicating that the drug combination of APC and doxorubicin has relatively low synergism in killing in KB-3-1 cells. For instance, the CI value all along the horizontal line labeled C is greater than 1 meaning that at the drug combination C (please refer to Table 3a for reference), not only is there no synergism, the drug combination has antagonism effect therein. However, it is shown that a lower concentration of APC together with a higher concentration of doxorubicin was sufficient to achieve a relatively higher synergistic effect but at low fractional kill of KB-3-1 cells. This is indicated by the darkened areas at the bottom left corner of FIG. 11.

Referring to FIG. 12, it is shown that at the drug combination C (please refer to Table 3b for reference) shown by the horizontal line labeled therewith, there is a relatively high level of synergism therein especially at a high concentration of the drug combination (at the right end of the horizontal line). This is shown by the white area situated next the label C. In other words, FIG. 12 of the Combination Studies shows that there are generally relatively high synergistic interactions for APC with doxorubicin on MDR KB-V1 cells, particularly at a concentration where the fractional kill is greater than 50% (i.e. right hand side of FIG. 12). The experiment also indicates that the synergistic effects for APC with doxorubicin on MDR KB-V1 cells are dose-independent of APC ranging from 2.0219 μM to 129.3996 μM. The KB-3-1 cells were used as control experiments and it is shown that there are little or no synergistic interactions under the same concentrations of APC ranging from 2.0219 μM to 129.3996 μM. Combination studies indicate that APC would produce synergistic activity to MDR cells, which is useful in engineering therapeutic strategies and pharmaceutical compositions incorporating APC for the treatment cancers. In addition to doxorubicin, other drugs including but not limited to anthracycline, Vinca alkaloid, epipodophyllotoxin and taxane may be used with APC and/or its variations in treating against cancer cells and particularly MDR cancer cells.

### Experiment 11

### Effects of Drug Combination Having the Crude Extracts and Doxorubicin

This experiment is similar to Experiment 10 in that the CI-isobologram by Chou and Talalay was similarly used to determine the Combination Index (CI) of a drug combination. However, APC is not used but crude extract of Peucedanum praeruptorum Dunn (in particularly the root thereof) having APC is used. The crude extract was prepared using conventional method to obtain extracts from herb well known to a skilled person in the field. In order to understand the synergistic effects of the crude extract with a conventional cancer therapeutic drug on drug-sensitive and MDR cells, various combinations of drug concentrations were similarly used in the combination studies (see Tables 5a to 6b).

Results and Discussion:

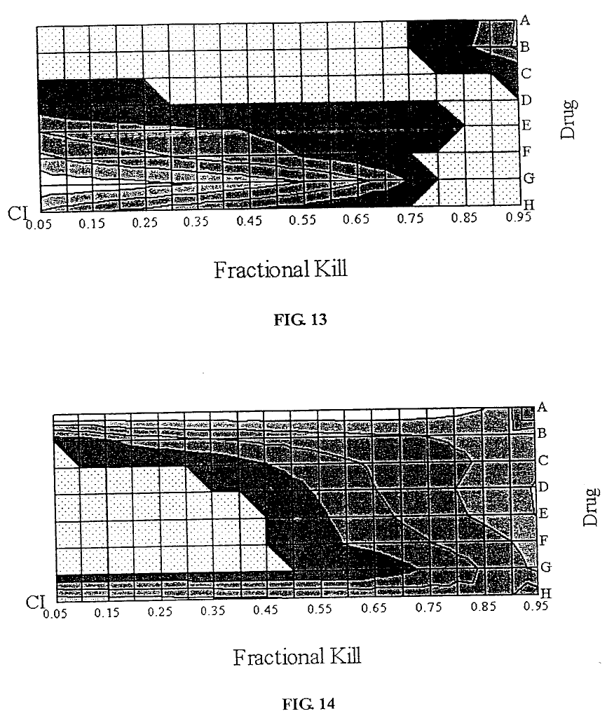

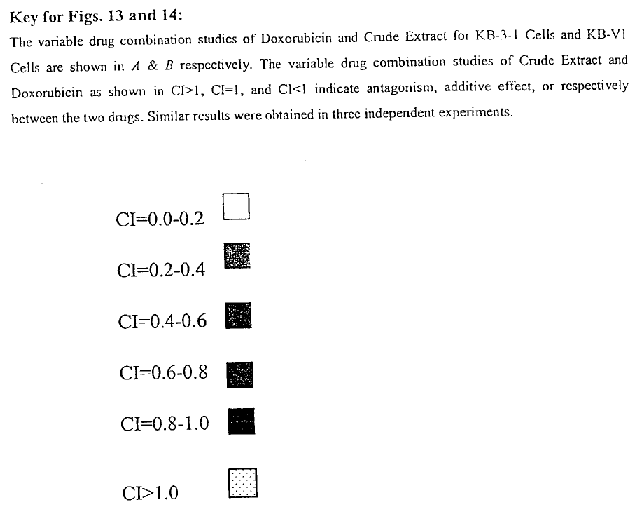

FIGS. 13 and 14 are graphs showing the variable drug combination studies of the crude extract and doxorubicin respectively. Similarly, the graphs are to be read in conjunction with the Key following the figures and Tables 5a to 6b.

As discussed above, a low CI value means that there is relatively high synergism in the drug combination. FIG. 13 shows that at a relatively high level of fractional kill of the KB-3-1 cells (e.g. fractional kill >0.9), the drug combinations D to H have a relatively high CI value (>1.0) indicating that each of these drug combinations has little synergism therein. FIG. 14 shows that at a relatively high level of fractional kill (e.g. fractional kill >0.9), all drug combinations A to H generally have a CI value of less than 1 indicating that each of these drug combinations has at least some synergism therein. In particular, there is a small complete island-shaped white area situated next the label D. It is thus indicated that at sufficiently high concentration of the drug combination D can effect a substantially total kill of KB-V1 cells and that there is relatively high synergism therein for the treatment against KB-V1 cells. Studying FIGS. 13 and 14 together, it can be concluded that the respective drug combinations A to H are more effective in treating MDR cancer cells such as KB-V1 than drug-sensitive KB-3-1. In other words, the crude extract equally has therapeutic activity in treating MDR cancer cells but with relatively lower effectiveness. The therapeutic activity results from the chemical compounds of APC and its variations.

### Experiment 12

### Reverse Transcription-PCR of MDR1

In this experiment, the effect of APC on KB-3-1 and KB-V1 cells is explored further. In particular, the effect of APC on P-glycoprotein (P-gp) and Actin as well as the mRNA thereof (i.e. MDR1 and β-Actin) in the cells are studied.

1 μg of total RNA isolated from cells using High Pure™ RNA Isolation Kits (Roche) was incubated with 100 ng random primers (Gibco BRL) and DEPC-treated water (total volume 10 μl) for 10 min at 65° C., then for 5 min on ice. Reverse transcription mixture contained 4 μl of 5× reaction buffer (50 mM Tris-HCl, 100 mM NaCl, 1 mM EDTA, 10 mM DTE, 0.05% polydocanol v/v, 50% glycerol v/v, pH 8.4), 2 μl 10 mM dNTP, 2 μl 100 mM dithiothreitol and 1 μl 50 units/μl Expand™ Reverse Transcriptase (Roche). The reaction lasted for 1.5 hours at 42° C. PCR amplifications were carried out using Expand™ Long Template PCR System (Roche). The primers used were cDNA sequences of p-glycoprotein (P-gp) SEQ ID NO. 1 (sense strand) and SEQ ID NO. 2 (anti-sense strand); cDNA sequences of β-actin SEQ ID NO. 3 (sense strand) and SEQ ID NO. 4 (anti-sense strand). Five stCi of μCi of [α-³²P]dATP was added to 25 μl amplification reaction mix. The PCR was performed in a Gene Cycler™ (Bio-Rad) for 27 cycles, consisting of 1 min at 94° C., 1 min at 57° C. and 1 min at 72° C. The samples were heated for 5 min at 94° C. before the first cycle, and the extension time was lengthened to 10 min during the last cycle. PCR products resulted therefrom were size-fractionated on 10% polyacrylamide gel. Autoradiography was carried out with BioMax film (Kodak). Drug-sensitive KB-3-1 was used as control experiments. Similar results were obtained in two independent experiments.

For protein analysis of P-gp and actin, cells were lysed in ice-cold lysis buffer (1% sodium deoxycholate, 0.1% SDS, 1% Triton X-100, 2 mM phenylmethylsulphonyl fluoride, 1% aprotinin). Total lysates were subjected to SDS/PAGE and electrotransferred to nitrocellulose membranes (Bio-Rad). Membranes were then incubated with anti-P-gp or anti-β-actin antibody (Calbiochem) in blocking buffer (10 mM Tris pH7.5, 100 mM NaCl, 0.1% Tween 20, 5% non-fat dry milk) for 1 hour at room temperature before incubation with horseradish-peroxidase-conjugated anti-(rabbit IgG) or anti-(mouse IgG) antibody (Gibco BRL) for 1 hour at room temperature. Immunobinding was detected by the ECL method (Amersham). β-actin polyclonal antibody (Oncogene Science, Uniondale, N.Y., USA) was used as internal standard. Drug-sensitive KB-3-1 was used as control experiments.

Results and Discussion:

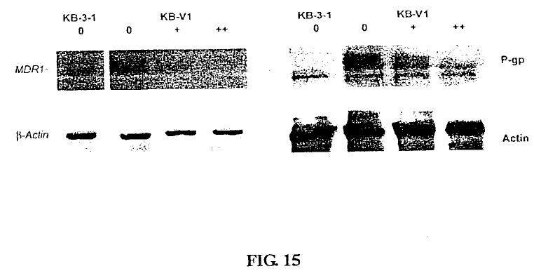

FIG. 15 shows the polyacrylamide gel images (on the left) of the MDR1, β-Actin in the cells. The value “0” indicates that the cells were not treated with any drug. The concentration of APC indicated by “+” and “++” means 1× and 2× dose of LD₅₀ respectively. β-Actin was used as internal standard for the experiment. It is shown that when the KB-3-1 and KB-V1 cells were treated with 1× and 2× dose of APC, the MDR1 was reduced significantly as indicated by the relatively faint bands. However, it is to be noted that β-Actin was not affected.

FIG. 15 also shows the Western Blotting (on the right) of P-gp and actin from the KB-3-1 and KB-V1 cells. Similarly, it is shown that the level of P-gp proteins of the cells treated with 1× and 2× dose of APC were lower indicated by the relatively faint bands thereof. The level of actin from the cells treated by APC was not affected.

Based on the above results, it can be concluded that APC is an inhibitor of MDR1/P-gp. The RT-PCR of MDR1 shows that MDR KB-V1 cells expressed high level of MDR1 relative to their drug-sensitive parent, KB-3-1 cells. The RT-PCR also clearly shows the down-regulations of the levels of MDR1 mRNA in MDR KB-V1 cells in a dose-dependent manner of APC. The Western blotting of P-gp shows that MDR KB-V1 cells expressed a higher level of P-gp relative to their drug-sensitive parent, KB-3-1 cells. The Western blotting also clearly shows the down-regulations of the levels of P-gp in MDR KB-V1 cells in a does-dependent manner of APC (FIG. 15).

The experiment suggests that APC down-regulates the levels of MDR1 mRNA, and inhibits the expression of P-gp in MDR KB-V-1 cells at a dose-dependent manner. It can therefore be concluded that APC can cause apoptosis in cancer cells in a preferred manner of programmed cell death by down regulating of the expression of MDR1 mRNA as well as P-gp proteins therein. The structural protein actin was not affected indicates that APC does not cause sudden cell death and/or rupture of the cell.

### Experiment 13

### Doxorubicin Accumulation Assays

In this experiment, the effect of APC on the accumulation of doxorubicin in the KB-3-1 and KB-V1 cells is studied. 5×10⁶ cells were collected from trypsinization and resuspended in 2 ml Hanks' balanced salt solution (HBSS) after APC pre-treatment at concentration of LD₅₀ for different time periods (i.e. 0, 2, 4 & 6 hours). Cells were incubated then with 2 μM doxorubicin as dye for 30 min at 37° C. in a humidified 5% CO₂ incubator. Doxorubicin content of the cells was determined by using Becton Dickinson Flow Cytometer (FACSCalibur) at excitation 488 nm/emission 600 nm. Data was collected with CellQuest Software and analysed with ModFitLT V2.0. Mean fluorescence intensities of 1×10⁴ drug-treated cells were quantified and compared with the APC untreated cells. Verapamil was used in place of APC as positive control. A negative control test was also performed. The mean fluorescence of 1×10⁴ cells pre-treated by verapamil and APC were quantified and compared with the APC untreated cells.

Results and Discussion:

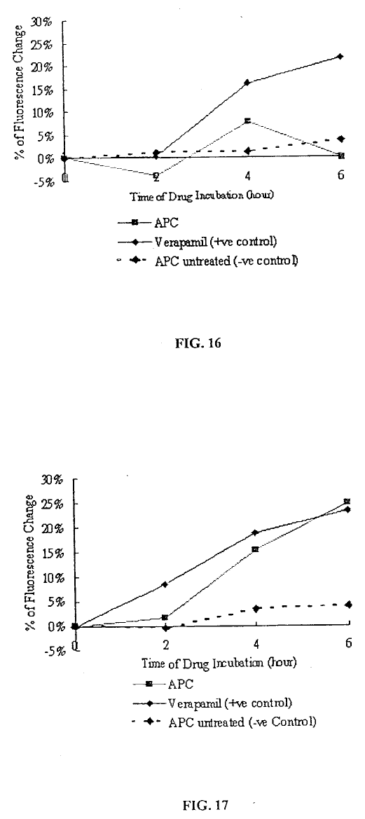

FIGS. 16 and 17 are graphs showing the accumulation of doxorubicin in KB-3-1 and KB-V1 cells respectively after treatment with APC. In FIG. 16, it is shown that there was a highest level of doxorubicin accumulated in the KB-3-1 cells after the pre-treatment with verapamil shown by the verapamil positive control. This is expected as verapamil is a drug which is known to turn off calcium pumps on cell surface, and it is believed that these pumps can remove drugs from the cells. It is indicated that once the cells were treated with verapamil, subsequent treatment with doxorubicin causes a higher level of accumulation of doxorubicin. This can be explained by the fact that once the calcium pumps on the cell surface are turned off, doxorubicin treating and entering the cells becomes accumulated therein. Referring to the APC curve in FIG. 17, it is shown that the cells pre-treated with APC have more doxorubicin accumulated therein when compared to the cells not having treated with APC or verapamil at all (see APC untreated negative control curve). However, APC is not as effective as verapamil in increasing and/or causing accumulation of doxorubicin in the cells.

In FIG. 17, the effect of pre-treatment on the accumulation of doxorubicin in the KB-V1 cells is illustrated. It is shown that pre-treatment of the cells with any of verapamil and APC has similar effect on the accumulation of doxorubicin therein. In particular, pre-treatment of any of verapamil and APC can cause a substantially higher accumulation of doxorubicin in the cells. This can be explained that, like verapamil, APC can effect of the down regulation of MDR1/P-gp associated with the calcium pumps. It is indicated in FIG. 17 that APC can increase the doxorubicin accumulation of MDR KB-V1 cells by approximately 25% after 6 hours incubation with APC and the effect of APC is comparable with verapamil. It is to be noted that a less significant effect of APC is seen on the increase in doxorubicin in the parent cells, KB-3-1 cells (see FIG. 16). This suggests that APC by itself or in association with other cancer therapeutics is more effective in treating against MDR cancer cells such as KB-V1 than drug sensitive cancer cells such as KB-3-1. It is also to be noted that the effect of APC on the increase of accumulation of intracellular cancer therapeutic drug such as doxorubicin in MDR KB-V1 cells is found to be time-dependent.

P-gp is ATP-dependent transporters mediating efflux cancer therapeutics from MDR tumor cells. The down regulation of the level of MDR1 mRNA by APC would inhibit the expression of P-gp. The results in this experiment and Experiment 12 together indicate that APC is an inhibitor of P-gp-mediated MDR.

Based on the above data, activity of MDR cancer cells can be modulated by APC by inhibition of P-gp expression. MDR KB-V1 cells have an over-expression of P-gp, thus sustaining a greater effect with APC treatment. On the other hand, drug-sensitive cells have no over-expression of P-gp thus sustaining a lesser effect by APC treatment.

Further Discussion

It can be concluded that APC and its variations have a mild cytotoxic effect in comparison with at least doxorubicin, a preferential induction of apoptosis to MDR cells, a synergistic effect with other drugs including doxorubicin with respect to MDR cells and a potent inhibitory effect to P-gp mediated MDR cells. APC and its variations are new and specific agents for the reversal of MDR in vitro.

In particular, the preferential killing of MDR cancer cells by APC has been studied in the present invention. It has been shown that the preferential killing of MDR cells by APC is correlated to the greater susceptibility to induction of apoptosis in MDR cells. The MDR cell lines had previously been shown to undergo apoptosis much more readily than sensitive cells when exposed to 2-deoxy-D-glucose (Bell, S. E., Quinn, D. M., Kellett, G. L., Warr, J. R., Br. J Cancer 78: 1464-1470, 1998) or inhibitors of glucosylceramide synthase (PDMP) and (PPPP) (Nicholson, K. M., Quinn, D. M., Kellett, G. L., Warr, J. R., British J Cancer, 81(3): 423-430, 1999). It was suggested that manipulation of glucosylceramide levels might be a fruitful way of causing the preferential killing of MDR cells. It is important to note that APC and its variations discussed in the present invention are members of the set of related chemical compounds, which is completely different class of agent from 2-deoxy-D-glucose, 1-phenyl-2-decanoylamino-3-morpholino-1-propanol (PDMP) and 1-phenyl-2-hexadecanoylamino-3-pyrrolidino-1-propano(PPPP). The experiments above indicate that MDR cells are preferentially more sensitive to the killing and induction of apoptosis by a wide range of stimuli. Although the mechanism by which APC preferentially eradicate MDR cancer cells is not completely known, the above experimental results are certainly sufficient to enable the engineering of new cancer therapeutics and treatment including combination-chemoprevention and therapeutic strategies.

MDR is recognized clinically as one of the most common causes of failure of cancer chemotherapy. Prolonged chemotherapy of cancer often results in the selective survival of cancer cells that are cross-resistance to a spectrum of structurally and functionally unrelated chemical drugs. Several mechanisms may account for the MDR of the cancer cells, including failure of the cells to undergoing apoptosis in response to chemotherapy, or failure of the drug to reach and/or affect its intracellular target. Successful anti-cancer treatments such as drugs, biologics, or radiation must hit their cellular targets and then causes some form of cellular alteration or damage. Rather than killing the cancer cell directly, it is believed that in most cases the damage inflicted by the anti-cancer agent triggers the process of programmed cell death, or apoptosis. Hence, resistance to multiple drugs could arise from cellular defense that broadly limit access of the agent to a cellular target or prevent the cell from entering apoptosis following injury.

Unless stated otherwise, all procedures were performed using standard protocols and following manufacturer′s instructions where applicable. Standard protocols for various techniques including PCR, molecular cloning, manipulation and sequencing, the manufacture of antibodies, epitope mapping and mimotope design, cell culturing and phage display, are described in texts such as McPherson, M. J. et al. (1991, PCR: A practical approach, Oxford University Press, Oxford), Sambrook, J. et al. (1989, Molecular cloning: a laboratory manual, Cold Spring Harbour Laboratory, New York), Huynh and Davies (1985, “DNA Cloning Vol I—A Practical Approach”, IRL Press, Oxford, Ed. D. M. Glover), Sanger, F. et al. (1977, PNAS USA 74(12): 5463-5467), Harlow, E. and Lane, D. (“Using Antibodies: A Laboratory Manual”, Cold Spring Habour Laboratory Press, New York, 1998), Jung, G. and Beck-Sickinger, A. G. (1992, Angew. Chem. Int. Ed. Eng., 31: 367-486), Harris, M. A. and Rae, I. F. (“General Techniques of Cell Culture”, 1997, Cambridge University Press, ISBN 0521 573645), “Phage Display of Peptides and Proteins: A Laboratory Manual” (Eds. Kay, B. K., Winter, J., and McCafferty, J., Academic Press Inc., 1996, ISBN 0-12-402380-0).

Reagents and equipment useful in, amongst others, the methods detailed herein are available from the likes of Amersham (www.amersham.co.uk), Boehringer Mannheim (www.boehringen-ingelheim.com), Clontech (www.clontech.com), Genosys (www.genosys.com), Millipore (www.millipore.com), Novagen (www.novagen.com), Perkin Elmer (www.perkinelmer.com), Pharmacia (www.pharmacia.com), Promega (www.promega.com), Qiagen (www.qiagen.com), Sigma (www sigma-aldrich.com) and Stratagene (www.Stratagene.com).

### Appendix

Determination of Combination Indices (CI)

A1. Median-Effect Principle

This is based on the mass action law, and it can be applied for multiple enzyme inhibitions by a simple graphical method. The median-effect equation is:

$$\frac{fa}{fu} = {( \frac{D}{Dm} )}^{m}$$

$$or$$

$$log ( \frac{fa}{fu} ) = m log ( D ) - m log ( Dm )$$

in which ‘fa’ is the fraction of cells affected by drugs and ‘fu’ is the fraction of cells unaffected by drug, i.e. fa+fu=1; ‘D’ is the dose , ‘Dm’ is the dose required for 50% effect equivalent to IC₅₀,LD₅₀ or ED₅₀ depending on the end-point of the experiment.

‘m’ is a coefficient (analogous to Hill coefficient of the Hill equation in the Ligand receptor analysis) of the sigmoidicity of the dose-effect curve; when m=1, m>1, and m<1 indicate Hyperbolic (first order kinetic or Michaelis-Menten type), S-shape (sigmoidal or higher order kinetic), and Negative sigmoidal dose-effect curves (allosteric system), respectively, for an inhibitory drug.

By plotting the Y=log(fa/fu) against X=log(D), anti-log at the x-intercept is equal to Dm and the slope is equal to m. Thus, the method takes into account both the potency (Dm), and shape (m) parameters.

A2. Combination Index Analysis

For a system of combined drugs, synergism is more than additive effect of individual drug and antagonism is a less than additive effect of individual drug. The additive effect of two drugs may not be the simple arithmetic sum of the effects of two drugs.

The Chou and Talalay (Chou T C; Talay P. Adv Enzyme Regul 1984 22:27-55) combination index CI-isobologram equation for two-drug combination is:

$$CI = \frac{{( D )}_{1}}{{( Dx )}_{1}} + \frac{{( D )}_{2}}{{( Dx )}_{2}} + \frac{{α ( D )}_{1} {( D )}_{2}}{{( Dx )}_{1} {( Dx )}_{2}}$$

α=0 for Mutually Exclusive or having same or similar modes of action; α=1 for Mutually Non-exclusive or having different or independent modes of action.

(Dx)₁ and (Dx)₂ is the dose of drug 1 and drug 2 alone required to achieve a certain cell killing rate (fa), whilst (D)₁ and (D)₂ is the dose of drug 1 and drug 2 in the combined drug to achieve a certain cell killing rate (fa).

Where, CI<1, CI=1, CI<1 indicate Synergism, Additive, and Antagonism effect, respectively.

This equation can be applied to determine synergism or antagonism even without the knowledge of mechanisms of action. In many drugs combinations, each drug may have more than one mode of the action, and synergism may be due to other than the direct mechanism of action i.e. membrane transport, drug resistance reversal, interference of metabolic activation or inactivation. This equation emphasizes the quantitative and results of drugs combination rather than the mechanism of synergistic or antagonistic interaction.

Firstly, the Din (Median Dose or LD₅₀) and m of each treatment was determined according to the Median-Effect Plot. The combined effect of two drugs was studied with mixture containing varying total amounts but a constant concentration ratio. Combination Indices (CI) were calculated at various fractional kill (fa).

A3. Determination of a 3-D Combination Indices

The CI-isobologram by Chou and Talalay was used to determine the Combination Indices (CI) of APC and doxorubicin. To have a comprehensive spectrum of understanding to the synergistic effects of APC, variable combinations of drug concentrations were used in the combination studies (see table below). The mutually non-exclusive equations were used to determine the CIs and each CI was calculated from the mean affected fraction at each drug ratio concentration.

KB-3-1 Cells

| | Doxorubicin | |
|---|---|---|
| Drug | (μM) | APC (μM) |
| A | 0.0108 | 129.3996 |
| B | 0.0216 | 129.3996 |
| C | 0.0431 | 129.3996 |
| D | 0.0862 | 129.3996 |
| E | 0.0862 | 64.6998 |
| F | 0.0862 | 32.3499 |
| G | 0.0862 | 16.1749 |
| H | 0.0862 | 8.0875 |
| I | 0.0862 | 4.0437 |
| J | 0.0862 | 2.0219 |

KB-V-1 Cells

| | Doxorubicin | |
|---|---|---|
| Drug | (μM) | APC (μM) |
| A | 1.0776 | 129.3996 |
| B | 2.1552 | 129.3996 |
| C | 4.3104 | 129.3996 |
| D | 8.6208 | 129.3996 |
| E | 8.6208 | 64.6998 |
| F | 8.6208 | 32.3499 |
| G | 8.6208 | 16.1749 |
| H | 8.6208 | 8.0875 |
| I | 8.6208 | 4.0437 |
| J | 8.6208 | 2.0219 |

The above two tables of data shows that the variable combinations of drug concentrations of doxorubicin and APC were prescribed as Drug A to Drug J for combination index and synergism studies.

It has been determined the Dm and m for each particular drug combination (Drug A to J) and the single drug of APC and Doxorubicin, by the following median effect equation.

$$\frac{fa}{fu} = {( \frac{D}{Dm} )}^{m}$$

$$or$$

$$D = {Dm ( \frac{fa}{1 - fa} )}^{\frac{1}{m}}$$

At a certain fractional kill (from 0% kill to 100% kill), C.I. is calculated from the following equation.

$$CI = \frac{{( D )}_{1}}{{( Dx )}_{1}} + \frac{{( D )}_{2}}{{( Dx )}_{2}} + \frac{{α ( D )}_{1} {( D )}_{2}}{{( Dx )}_{1} {( Dx )}_{2}}$$

$$or$$

$$CI = ( \frac{{w}_{1}}{{w}_{1} + {w}_{2}} ) \frac{{( D )}_{1}}{{( Dx )}_{1}} + ( \frac{{w}_{2}}{{w}_{1} + {w}_{2}} ) \frac{{( D )}_{2}}{{( Dx )}_{2}} + \\ \frac{α ( \frac{{w}_{1}}{{w}_{1} + {w}_{2}} ) {( D )}_{1} ( \frac{{w}_{2}}{{w}_{1} + {w}_{2}} ) {( D )}_{2}}{{( Dx )}_{1} {( Dx )}_{2}}$$

A 3-D CI (Z-axis) graph could be plotted for Drug A to I (Y-axis) at different fractional kill (X-axis). The combination of the lowest value in CI and highest value in fractional kill is ideal.

1. A chemical compound of the following formula 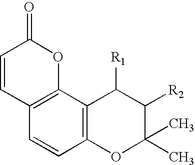
wherein R1 and R2 may be the same or different and are selected from a saturated or unsaturated acid;
or a physiologically acceptable salt thereof.

2. The chemical compound according to claim 1 wherein said R1 and R2 are selected from a saturated or unsaturated acid including two to five carbons.

3. The chemical compound according to claim 2 wherein said R1 and R2 are selected from the group consisting of —OCOCH₃ and —OCOCCH₃═CHCH₃.

4. The chemical compound according to claim 1 wherein said chemical compound is of following formula 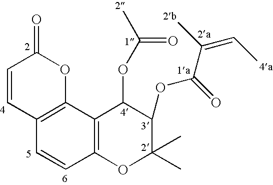
or a physiologically acceptable salt thereof.

5. The chemical compound according to claim 1, wherein said compound is “cis-3′-angeloyl-4′-acetoxy-khellactone”.

6. The chemical compound according to claim 1 wherein said compound is selected from the group consisting of “cis-3′-acetoxy-4′-angeloyl-khellactone”, “trans-3′-angeloyl-4′-acetoxy-khellactone”, and “trans-3′-acetoxy-4′-angeloyl-khellactone”.

7. The chemical compound of claim 1 isolated from a natural source.

8. The chemical compound of claim 2 isolated from a natural source.

9. The chemical compound of claim 3 isolated from a natural source.

10. The chemical compound of claim 4 isolated from a natural source.

11. The chemical compound of claim 5 isolated from a natural source.

12. The chemical compound of claim 6 isolated from a natural source.

13. The chemical compound according to claim 1 wherein said natural source is the plant of Peucedanum praeruptorum Dunn (Umbelliferae).

14. The chemical compound according to claim 2 wherein said natural source is the plant of Peucedanum praeruptorum Dunn (Umbelliferae).

15. The chemical compound according to claim 3 wherein said natural source is the plant of Peucedanum praeruptorum Dunn (Umbelliferae).

16. The chemical compound according to claim 4 wherein said natural source is the plant of Peucedanum praeruptorum Dunn (Umbelliferae).

17. The chemical compound according to claim 5 wherein said natural source is the plant of Peucedanum praeruptorum Dunn (Umbelliferae).

18. The chemical compound according to claim 6 wherein said natural source is the plant of Peucedanum praeruptorum Dunn (Umbelliferae).

19. A composition comprising at least one of the chemical compounds of the following formula 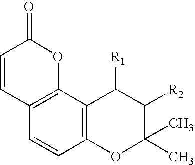
wherein R1 and R2 may be the same or different and are selected from a saturated or unsaturated acid;
or a physiologically acceptable salt thereof;
a chemical substance selected from a group consisting of doxorubicin, anthracycline, Vinca alkaloid, epipodophyllotoxin and taxane; and
optionally, one or more physiologically acceptable adjuvants, diluents, excipients or carriers.

20. The composition according to claim 19 wherein said R1 and R2 are selected from a saturated or unsaturated acid including two to five carbons.

21. The composition according to claim 20 wherein said R1 and R2 are selected from the group consisting of —OCOCH₃ and —OCOCCH₃═CHCH₃.

22. The composition according to claim 1 wherein the chemical compound is of the following formula

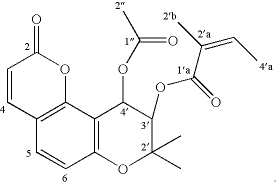

23. The composition according to claim 19, wherein said chemical compound is “cis-3′-angeloyl-4′-acetoxy-khellactone”.

24. The composition according to claim 19 wherein said chemical compounds are selected from the group consisting of “cis-3′-acetoxy-4′-angeloyl-khellactone”, “trans-3′-angeloyl-4′-acetoxy-khellactone”, and “trans-3′-acetoxy-4′-angeloyl-khellactone”.

25. A method for treatment of cancer comprising administering a therapeutically effective amount of at least one chemical compound of the following formula 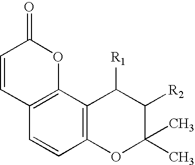
wherein R1 and R2 may be the same or different and are selected from a saturated or unsaturated acid;
or a physiologically acceptable salt of said compound.

26. The method for treatment of cancer according to claim 25 wherein said R1 and R2 are selected from a saturated or unsaturated acid including two to five carbons.

27. The method for treatment of cancer according to claim 26 wherein said R1 and R2 are selected from the group consisting of —OCOCH₃ and —OCOCCH₃═CHCH₃.

28. The method for treatment of cancer according to claim 25, wherein the compound is of the following formula

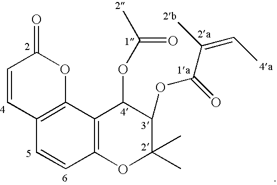

29. The method for treatment of cancer according to claim 25, wherein said compound is “cis-3′-angeloyl-4′-acetoxy-khellactone”.

30. The method for treatment of cancer according to claim 25, wherein said compound is selected from the group consisting of “cis-3′-acetoxy-4′-angeloyl-khellactone”, “trans-3′-angeloyl-4′-acetoxy-khellactone”, and “trans-3′-acetoxy-4′-angeloyl-khellactone”.

31. The method for treatment of cancer according to claim 25, wherein said compound is administered to treat cancer caused by multi-drug resistant (MDR) cancer cells.

32. The method for treatment of cancer according to claim 26, wherein said compound is administered to treat cancer caused by multi-drug resistant (MDR) cancer cells.

33. The method for treatment of cancer according to claim 27, wherein said compound is administered to treat cancer caused by multi-drug resistant (MDR) cancer cells.

34. The method for treatment of cancer according to claim 28, wherein said compound is administered to treat cancer caused by multi-drug resistant (MDR) cancer cells.

35. The method for treatment of cancer according to claim 29, wherein said compound is administered to treat cancer caused by multi-drug resistant (MDR) cancer cells.

36. The method for treatment of cancer according to claim 30, wherein said compound is administered to treat cancer caused by multi-drug resistant (MDR) cancer cells.

37. The method for treatment of cancer according to claim 25, wherein said compound is administered to cause apoptosis in cancer cells.

38. The method for treatment of cancer according to claim 26, wherein said compound is administered to cause apoptosis in cancer cells.

39. The method for treatment of cancer according to claim 27, wherein said compound is administered to cause apoptosis in cancer cells.

40. The method for treatment of cancer according to claim 28, wherein said compound is administered to cause apoptosis in cancer cells.

41. The method for treatment of cancer according to claim 29, wherein said compound is administered to cause apoptosis in cancer cells.

42. The method for treatment of cancer according to claim 30, wherein said compound is administered to cause apoptosis in cancer cells.

43. The method for treatment of cancer according to claim 30, wherein said compound is isolated from the plant, Peucedanum praeruptorum Dunn (Umbelliferae).

44. A method for treatment of cancer comprising administering a therapeutically effective amount of a composition including at least one chemical compound of the following formula 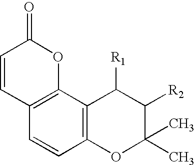
wherein R1 and R2 may be the same or different and are selected from a saturated or unsaturated acid;
or a physiologically acceptable salt of said compound;
a chemical substance selected from a group consisting of doxorubicin, anthracycline, Vinca alkaloid, epipodophyllotoxin and taxane; and
optionally, one or more physiologically acceptable adjuvants, diluents, excipients or carriers.

45. The method for treatment of cancer according to claim 44, wherein said R1 and R2 are selected from a saturated or unsaturated acid including two to five carbons.

46. The method for treatment of cancer according to claim 45, wherein said R1 and R2 are selected from the group consisting of —OCOCH₃ and —OCOCCH₃═CHCH₃.

47. The method for treatment of cancer according to claim 44, wherein the chemical compound is of the following formula

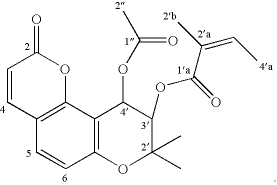

48. The method for treatment of cancer according to claim 44, wherein said chemical compound is “cis-3′-angeloyl-4′-acetoxy-khellactone”.

49. The method for treatment of cancer according to claim 44, wherein said chemical compounds are selected from the group consisting of “cis-3′-acetoxy-4′-angeloyl-khellactone”, “trans- 3′-angeloyl-4′-acetoxy-khellactone”, and “trans-3′-acetoxy-4′-angeloyl-khellactone”.

50. A method of isolating one or more chemical compounds of the following formula 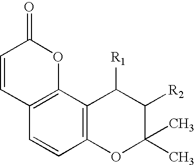
wherein R1 and R2 may be the same or different and are selected from a saturated or unsaturated acid;
from the plant of Peucedanum praeruptorum Dunn (Umbelliferae) comprising:
a) drying said plant;
b) treating said plant with 30 to 100 wt % of an alcohol for at least one hour to produce an extract therefrom;
c) filtering said extract by a suitable filter paper;
d) removing said alcohol from said filtered extract to produce a first residue;
e) obtaining organic fraction of said first residue by extraction with chloroform to produce a second residue;
f) purifying said second residue by column chromatography; and
g) producing crystals comprising at least one of said chemical compounds by re-crystallizing said purified second residue.

51. The method according to claim 50 further comprising powdering said plant.

52. The method according to claim 50 wherein said alcohol is selected from a group including methanol, ethanol and propanol.

53. The method according to claim 50 wherein said filter paper is a No. 2 filter paper.

54. The method according to claim 50 wherein said chemical compounds comprise substantially (+)-cis-3′-angeloyl-4′-acetoxy-khellactone” and (−)-cis-3′-angeloyl-4′-acetoxy-khellactone.


SEQUENCE LISTING (4 sequences)

SEQ ID NO: 1 - DNA, 20 bp, Peucedanum praerutorum, cDNA
cccatcattgcaatagcagg

SEQ ID NO: 2 - DNA, 20 bp, Peucedanum praerutorum, cDNA
gttcaaacttctgctcctga

SEQ ID NO: 3 - DNA, 27 bp, Peucedanum praerutorum, cDNA
gatgatatcgccgcgctcgtcgtcgac

SEQ ID NO: 4 - DNA, 27 bp, peucedanum praerutorum, cDNA
agccaggtccagacgcaggatggcatg